(19) United States
(12) Patent Application Publication
(10) Pub. No.: US 2023/0158049 A1
(43) Pub. Date: May 25, 2023

(54) COMPOSITIONS AND METHODS FOR TREATING VIRUSES

(71) Applicants: Elias Reichel, Weston, MA (US); University of Virginia Patent Foundation, Charlottesville, VA (US)

(72) Inventors: Paul Andrew Yates, Charlottesville (US); Elias Reichel, Weston (US); Ashton Leone, Charlottesville (US)

(21) Appl. No.: 17/917,407

(22) PCT Filed: Apr. 6, 2021
(86) PCT No.: PCT/US2021/025986

§ 371 Date: Oct. 6, 2022

Related U.S. Application Data
(60) Provisional application No. 63/005,998, filed on Apr. 6, 2020.

(51) Int. Cl. A61K 31/65; A61P 31/14; A61K 33/30

(52) U.S. Cl. CPC A61K 31/65; A61P 31/14; A61K 33/30

(57) ABSTRACT

Disclosed are compositions and methods that can be used for the prevention, mitigation, and/or prophylactic treatment of a viral infection, such but not limited to a coronavirus infection, such as but not limited to a COVID-19 infection. In some embodiments, a chemically modified tetracycline (CMT) derivative for the prevention, mitigation and/or prophylactic treatment of a viral infection is provided. In some embodiments, the CMT derivative lacks anti-microbial activity; comprises a phenol ring; and/or comprises a chemical structure sufficient to chelate and/or bind a divalent cation. In some embodiments, the divalent cation comprises Zn²⁺. In some embodiments, the viral infection is a coronavirus infection. In some embodiments, the viral infection is a COVID-19 infection.

### CROSS REFERENCE TO RELATED APPLICATIONS

This application claims benefit of U.S. Provisional Application Ser. No. 63/005,998, filed Apr. 6, 2020, the disclosure of which is incorporated herein by reference in its entirety.

### TECHNICAL FIELD

The presently disclosed subject matter is directed to compositions and methods for treating viruses, including coronaviruses.

### BACKGROUND

SARS-CoV-2, the causative agent for a COVID-19 infection, is a newly emerged coronavirus for which vaccines have only recently become available and for which there is no proven prophylactic treatment. While vaccines are available, there continues to be an ongoing need for prevention of infection through public health, clinical testing, behavioral, and pharmacological approaches. This is particularly true for health care workers who risk daily exposure to COVID-19 positive patients and community contacts. Unfortunately, personal protection equipment (PPE) has been shown insufficient in and of itself, with 2-6% of healthcare workers routinely exposed to COVID-19 patients becoming infected themselves.

What is needed, then, are new therapeutic agents, compositions and methods for treating viruses, including for example coronaviruses and COVID-19.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

Provided in some embodiments of the presently disclosed subject matter is a chemically modified tetracycline (CMT) derivative. In some embodiments, the CMT derivative is for the treatment of a viral infection, such as the prevention, mitigation and/or prophylactic treatment of a viral infection. In some embodiments, the CMT derivative lacks anti-microbial activity; comprises a phenol ring; and/or comprises a chemical structure sufficient to chelate and/or bind a divalent cation. In some embodiments, the divalent cation comprises Zn²⁺. In some embodiments, the viral infection is a coronavirus infection. In some embodiments, the viral infection is a COVID-19 infection. In some embodiments, the CMT is disposed in an effective amount in an excipient or a pharmaceutically acceptable carrier.

In some embodiments, the CMT derivative can have antimicrobial activity due to a dosing at or above a minimum inhibitory concentration. In some embodiments, the CMT derivative lacks antimicrobial activity due to deletion of a C4 dimethylamino. In some embodiments, the CMT derivative lacks antimicrobial activity due to dosing below a minimum inhibitory concentration. In some embodiments, the phenol ring comprises a diethylamino group to enhance scavenging of reactive oxygen species.

In some embodiments, the chemical structure comprises the following

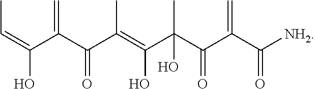 [SMILES: C=C(C(N)=O)C(=O)C(C)(O)/C(O)=C(\C)C(=O)C(=C)/C(O)=C\C]

structure:

In some embodiments, the CMT derivative comprises doxycycline.

In some embodiments, the CMT derivative is given at a dose of about 40 mg per day or less, optionally less than about 50 mg per day, less than about 60 mg per day, less than about 70 mg per day, less than about 80 mg per day, less than about 90 mg per day, less than about 100 mg per day, or less than about 200 mg per day.

In some embodiments, the CMT derivative is configured to inhibit metalloproteinases (MMPs). In some embodiments, the MMP is MMP-9. In some embodiments, the MMP is any MMP implicated in respiratory distress syndrome (ARDS). In some embodiments, the CMT derivative is configured to inhibit IL6 and other cytokine production. In some embodiments, the CMT derivative is configured to inhibit Papain-like protease (PLpro). In some embodiments, the inhibition of PLpro inhibits proteolytic cleavage of a replicase polyprotein needed for viral replication. In some embodiments, the replicase polyprotein is non-structural proteins 1, 2 and/or 3 (Nsp1, Nsp2 and/or Nsp3).

In some embodiments, the CMT derivative is configured to inhibit and/or bind 3C-like main protease (3CLpro) and/or Nsp5. In some embodiments, the inhibition and/or binding of 3CLpro and/or Nsp5 prevents viral replication.

In some embodiments, the CMT derivative is configured to bind a divalent cation and transport the divalent cation intracellularly. In some embodiments, transporting divalent cations intracellularly increases an intracellular concentration of the cation to thereby inhibit viral replication.

Provided in accordance with some embodiments of the presently disclosed subject matter is a method of treating a viral infection in a subject, including preventing, mitigating and/or prophylactically treating a viral infection in a subject. In some embodiments, the method comprising administering to a subject in need thereof an effective amount of a CMT derivative. In some embodiments, the CMT derivative lacks anti-microbial activity; comprises a phenol ring; and/or comprises a chemical structure sufficient to chelate and/or bind a divalent cation. In some embodiments, the divalent cation comprises Zn²⁺. In some embodiments, the viral infection is a coronavirus infection. In some embodiments, the viral infection is a COVID-19 infection.

In some embodiments, the CMT derivative can have antimicrobial activity due to a dosing at or above a minimum inhibitory concentration. In some embodiments, the effective amount comprises a dose of the CMT derivative sufficient to inhibit activation of gut microbiome inflammatory cells, wherein the dose of the CMT derivative is below a minimum inhibitory concentration for antimicrobial activity. In some embodiments, the CMT derivative is administered at a dose of about 40 mg per day or less, optionally less than about 50 mg per day, less than about 60 mg per day, less than about 70 mg per day, less than about 80 mg per day, less than about 90 mg per day, less than about 100 mg per day, or less than about 200 mg per day.

In some embodiments, the CMT derivative lacks antimicrobial activity due to deletion of a C4 dimethylamino. In some embodiments, the CMT derivative lacks antimicrobial activity due to dosing below a minimum inhibitory concentration. In some embodiments, the phenol ring comprises a diethylamino group to enhance scavenging of reactive oxygen species.

In some embodiments, the chemical structure comprises the following

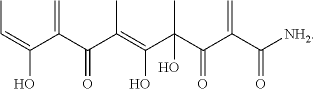 [SMILES: C=C(C(N)=O)C(=O)C(C)(O)/C(O)=C(\C)C(=O)C(=C)/C(O)=C\C]

structure:

In some embodiments, the CMT derivative comprises doxycycline, or a pharmaceutically acceptable salt thereof.

In some embodiments, the subject is a human subject. In some embodiments, the human subject is suffering from a coronavirus infection.

In some embodiments, the method further comprises administering to the subject a dose of a divalent cation, such as but not limited to Zn²⁺. In some embodiments, the divalent cation or Zn²⁺ is administered at a dosage of about 4 mg/day to about 50 mg/day, including about 4 mg/day to about 40 mg/day.

In some embodiments, the CMT derivative is configured to inhibit metalloproteinases (MMPs). In some embodiments, the MMP is MMP-9. In some embodiments, the MMP is any MMP implicated in respiratory distress syndrome (ARDS). In some embodiments, the CMT derivative is configured to inhibit IL6 and other cytokine production. In some embodiments, the CMT derivative is configured to inhibit Papain-like protease (PLpro). In some embodiments, the inhibition of PLpro inhibits proteolytic cleavage of a replicase polyprotein needed for viral replication. In some embodiments, the replicase polyprotein is non-structural proteins 1, 2 and/or 3 (Nsp1, Nsp2 and/or Nsp3).

In some embodiments, the CMT derivative is configured to inhibit and/or bind 3C-like main protease (3CLpro) and/or Nsp5. In some embodiments, the inhibition and/or binding of 3CLpro and/or Nsp5 prevents viral replication.

In some embodiments, the CMT derivative is configured to bind a divalent cation and transport the divalent cation intracellularly. In some embodiments, transporting divalent cations intracellularly increases an intracellular concentration of the cation to thereby inhibit viral replication.

Provided in accordance with some embodiments of the presently disclosed subject matter is a composition for administration to a subject, the composition comprising a chemically modified tetracycline (CMT) derivative. In some embodiments, the CMT derivative lacks anti-microbial activity, comprises a phenol ring, and/or comprises a chemical structure sufficient to chelate and/or bind a divalent cation. In some embodiments, the divalent cation is Zn²⁺. In some embodiments, the CMT derivative is included in the composition at a concentration sufficient to provide a dose of about 40 mg per day or less, optionally less than about 50 mg per day, less than about 60 mg per day, less than about 70 mg per day, less than about 80 mg per day, less than about 90 mg per day, less than about 100 mg per day, or less than about 200 mg per day, when administered to a subject. In some embodiments, the CMT derivative can have antimicrobial activity due to a dosing at or above a minimum inhibitory concentration.

In some embodiments, the composition further comprises a source of a divalent cation. In some embodiments, the divalent cation is Zn²⁺. In some embodiments, the divalent cation or Zn²⁺ is included in the composition at a concentration sufficient to provide a dose of about 4 mg/day to about 50 mg/day when administered to a subject, including about 4 mg/day to about 40 mg/day.

In some embodiments, the composition is configured to treat a viral infection, such as to prevent, mitigate and/or prophylactically treat a viral infection, when administered to a subject. In some embodiments, the viral infection is a coronavirus infection. In some embodiments, the coronavirus infection is a COVID-19 infection.

In some embodiments, the CMT derivative lacks antimicrobial activity due to deletion of a C4 dimethylamino. In some embodiments, the CMT derivative lacks antimicrobial activity due to dosing below a minimum inhibitory concentration. In some embodiments, the CMT derivative comprises doxycycline, or a pharmaceutically acceptable salt thereof. In some embodiments, the chemical structure comprises the following structure:

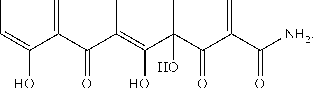 [SMILES: C=C(C(N)=O)C(=O)C(C)(O)/C(O)=C(\C)C(=O)C(=C)/C(O)=C\C]

In some embodiments, the composition further comprises an excipient or a pharmaceutically acceptable carrier.

In some embodiments, the CMT derivative is configured to inhibit metalloproteinases (MMPs). In some embodiments, the MMP is MMP-9. In some embodiments, the MMP is any MMP implicated in respiratory distress syndrome (ARDS). In some embodiments, the CMT derivative is configured to inhibit IL6 and other cytokine production. In some embodiments, the CMT derivative is configured to inhibit Papain-like protease (PLpro). In some embodiments, inhibition of PLpro prevents proteolytic cleavage of a replicase polyprotein needed for viral replication. In some embodiments, the replicase polyprotein is non-structural proteins 1, 2 and/or 3 (Nsp1, Nsp2 and/or Nsp3).

In some embodiments, the CMT derivative is configured to inhibit and/or bind 3C-like main protease (3CLpro) and/or Nsp5. In some embodiments, inhibition and/or binding of 3CLpro and/or Nsp5 prevents viral replication.

In some embodiments, the CMT derivative is configured to bind a divalent cation and transport the divalent cation intracellularly. In some embodiments, transporting divalent cations intracellularly increases an intracellular concentration of the cation to thereby inhibit viral replication.

Provided in accordance with some embodiments of the presently disclosed subject matter is a chemically modified tetracycline (CMT) derivative for the treatment of a viral infection, such as for the prevention, mitigation and/or prophylactic treatment of a viral infection. In some embodiments, the CMT derivative comprises a phenol ring; and comprises a chemical structure sufficient to chelate and/or bind a divalent cation. In some embodiments, the divalent cation is Zn²⁺.

Accordingly, it is an object of the presently disclosed subject matter to provide compositions and methods for treating viruses, including coronaviruses. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description and Examples.

### DETAILED DESCRIPTION

The presently disclosed subject matter provides in some embodiments compositions and methods that can be used for the treatment of a viral infection, such as but not limited to, prevention, mitigation, and/or prophylactic treatment of a viral infection, such but not limited to, a coronavirus infection, such as but not limited to a COVID-19 infection. In some embodiments, a chemically modified tetracycline (CMT) derivative for the treatment of a viral infection, such as but not limited to, for the prevention, mitigation and/or prophylactic treatment of a viral infection, is provided. In some embodiments, the CMT derivative lacks anti-microbial activity; comprises a phenol ring; and/or comprises a chemical structure sufficient to chelate and/or bind a divalent cation. In some embodiments, the divalent cation comprises Zn²⁺. In some embodiments, the viral infection is a coronavirus infection. In some embodiments, the viral infection is a COVID-19 infection. In some embodiments, the CMT derivative can have antimicrobial activity due to a dosing at or above a minimum inhibitory concentration.

Doxycycline is a CMT derivative and is an attractive candidate as a repurposed drug in the treatment of COVID-19 infection. As disclosed herein, anti-microbial and sub-anti-microbial doxycycline can offer substantial benefits as a treatment for viral infections, including coronavirus infection, and/or as a preventative agent against viral and coronavirus infections.

### I. DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed methods.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

As used herein, the term “subject” refers to an individual (e.g., human, animal, or other organism) to be assessed, evaluated, and/or treated by the methods or compositions of the presently disclosed subject matter. Subjects include, but are not limited to, mammals (e.g., murines, simians, equines, bovines, porcines, canines, felines, and the like), and includes humans. As used herein, the terms “subject” and “patient” are used interchangeably, unless otherwise noted.

As used herein, the terms “effective amount” and “therapeutically effective amount” are used interchangeably and refer to the amount that provides a therapeutic effect, e.g., an amount of a composition that is effective to treat or prevent pathological conditions in a subject.

As used herein, the term “adjuvant” as used herein refers to an agent which enhances the pharmaceutical effect of another agent.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids. The term “compound” is used interchangeably with “active ingredient.”

As used herein, a “derivative” of a compound refers to a chemical compound that can be produced from another compound of similar structure in one or more steps, such as in replacement of H by an alkyl, acyl, or amino group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “modulate”, as used herein, refers to changing the level of an activity, function, or process. The term “modulate” encompasses both inhibiting and stimulating an activity, function, or process.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in an animal. In some embodiments, a pharmaceutically acceptable carrier is pharmaceutically acceptable for use in a human.

As used herein, the term “pharmaceutically acceptable” ester or salt means an ester or salt form of the active ingredient which is compatible with any other ingredients of the pharmaceutical composition, and which is not deleterious to the subject to which the composition is to be administered.

The term “standard”, as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. For example, a prophylactic treatment can prevent a COVID-19 infection or decrease in severity of disease of those subjects who become COVID-19 positive. A “therapeutic” treatment or a “mitigation” is a treatment administered to a subject who exhibits signs of pathology for the purpose of diminishing or eliminating those signs.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. In some embodiments, the alkyl group is “lower alkyl.” “Lower alkyl” refers to an alkyl group having 1 to about 8 carbon atoms (i.e., a C₁₋₈ alkyl), e.g., 1, 2, 3, 4, 5, 6, 7, or 8 carbon atoms. In some embodiments, the alkyl is “higher alkyl.” “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic moiety that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, carbonyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to aryl groups wherein at least one atom of the backbone of the aromatic ring or rings is an atom other than carbon. Thus, heteroaryl groups have one or more non-carbon atoms selected from the group including, but not limited to, nitrogen, oxygen, and sulfur.

As used herein, the term “acyl” refers to an organic carboxylic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The terms “heterocycle” or “heterocyclic” refer to cycloalkyl groups (i.e., non-aromatic, cyclic groups as described hereinabove) wherein one or more of the backbone carbon atoms of a cyclic ring is replaced by a heteroatom (e.g., nitrogen, sulfur, or oxygen). Examples of heterocycles include, but are not limited to, tetrahydrofuran, tetrahydropyran, morpholine, dioxane, piperidine, piperazine, and pyrrolidine.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Alkoxyl” or “alkoxy” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangeably with “alkoxyl”.

“Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

The term “amino” refers to the —NR′R″ group, wherein R′ and R″ are each independently selected from the group including H and substituted and unsubstituted alkyl, cycloalkyl, heterocycle, aralkyl, aryl, and heteroaryl. In some embodiments, the amino group is —NH₂.

The term “carbonyl” refers to the —(C═O)— or a double bonded oxygen substituent attached to a carbon atom of a previously named parent group.

The term “carboxyl” refers to the —COOH group.

The terms “halo”, “halide”, or “halogen” as used herein refer to fluoro, chloro, bromo, and iodo groups.

The terms “hydroxyl” and “hydroxy” refer to the —OH group.

The term “oxo” refers to a compound described previously herein wherein a carbon atom is replaced by an oxygen atom.

The term “cyano” refers to the —CN group.

The term “nitro” refers to the —NO₂ group.

### II. REPRESENTATIVE COMPOSITIONS AND METHODS

Tetracycline derivatives are a therapy candidate for coronaviruses, given their ability to target many of these identified pathways that may contribute to the disease. In particular, tetracyclines are known to reduce reactive oxygen species, inhibit matrix metalloproteinase's (MMPs) that are involved in the breakdown of the barrier between the RPE and Bruch's membrane, inhibit caspase activation and thereby prevent cell death, prevent complement activation, and inhibit cytokine production through their effects on microglia and T-cell activation. The structural formula for tetracycline is as follows:

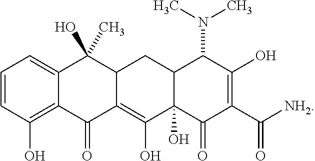 [SMILES: CN(C)[C@@H]1C(O)=C(C(N)=O)C(=O)[C@@]2(O)C(O)=C3C(=O)c4c(O)cccc4[C@@](C)(O)C3CC12]

There is very limited human data on the potential benefits of tetracycline derivatives. Among the tetracycline derivatives, doxycycline is a possible candidate for treatment of various conditions. However, as disclosed herein, submicrobial doxycycline can potentially offer substantial benefits as a treatment for viral infections, including coronavirus infection, and/or as a preventative agent against viral and coronavirus infections. The structural formula for doxycycline is as follows:

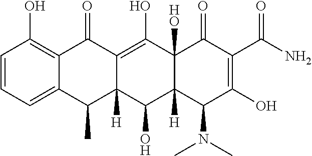 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

In some embodiments, doxycycline can provide one or more of the following benefits with respect to viral infections, including coronavirus infections.

1) inhibits metalloproteinases (MMPs), in particular MMP-9, which can be implicated in respiratory distress syndrome (ARDS).

2) inhibits IL6 and other cytokine production which can be involved in the inflammatory response to doxycycline.

3) PLpro is responsible for proteolytic cleavage of the replicase polyprotein to release non-structural proteins 1, 2 & 3 (Nsp1, Nsp2 and Nsp3) need for viral replication. Computational screening shows doxycycline as a potential PLpro inhibitor.

4) 3C-like main protease (3CLpro) or Nsp5 is required in the virus lifecycle. Computational screening again shows doxycycline can bind to 3CLpro.

5) Doxycycline is an ionophore and binds divalent cations (including Zn²⁺), much like plaquenil (hydroxychloroquine). Zinc has an inhibitory effect on the replication of coronaviruses, inhibiting replicase proteins and RNA dependent RNA polymerase activity. A zinc supplement in combination with doxycycline can in some embodiments allow doxycycline to help transport zinc (Zn) intracellularly to increase intracellular concentration to inhibit viral replication. Alternatively, the zinc can be taken out of sequence with the doxycycline to prevent zinc interference with systemic absorption of the doxycycline. This dosing regimen can eliminate chelation of zinc by doxycycline in the GI tract, allowing time for absorption of both for those subjects on both.

These features are all available with low dose doxycycline. In some embodiments such a treatment with low dose doxycycline or derivative can be substantially non-toxic treatment as it would not be anti-microbial. Thus, in some aspects, doxycycline can help decrease initial infection as well as subsequent progression of disease in infected patients.

In some embodiments provided herein are chemically modified tetracycline (CMT) derivatives. In some embodiments, the CMT derivatives are for the treatment of a viral infection, such as but not limited to the prevention, mitigation and/or prophylactic treatment of a viral infection. The CMT derivatives can optionally lack anti-microbial activity. The CMT derivative can comprise a phenol ring and/or can comprise a chemical structure sufficient to chelate and/or bind a divalent cation, optionally Zn²⁺. In some embodiments, the CMT derivative is disposed in an effective amount in an excipient or a pharmaceutically acceptable carrier.

The viral infection can be a coronavirus infection, optionally a COVID-19 infection. The CMT derivative can lack antimicrobial activity due to deletion of a C4 dimethylamino. The CMT derivative can lack antimicrobial activity due to dosing below a minimum inhibitory concentration. In some embodiments, the CMT derivative can have antimicrobial activity due to a dosing at or above a minimum inhibitory concentration. The phenol ring can comprise a diethylamino group to enhance scavenging of reactive oxygen species. The chemical structure can comprise the following structure:

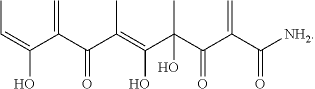 [SMILES: C=C(C(N)=O)C(=O)C(C)(O)/C(O)=C(\C)C(=O)C(=C)/C(O)=C\C]

In some embodiments, the CMT derivative can be minocycline, tigecycline, lymecycline, demeclocycline, oxytetracycline, rolitetracycline, or doxycycline.

In some embodiments, an effective amount of a CMT derivative of the presently disclosed subject matter is provided, such as in an excipient or a pharmaceutically acceptable carrier. In some embodiments, the effective amount comprises a dose of the CMT derivative sufficient to inhibit activation of gut microbiome inflammatory cells, wherein the dose of the CMT derivative is below a minimum inhibitory concentration for antimicrobial activity. In some embodiments, the effective amount of the CMT derivative is an amount where the CMT derivative can have antimicrobial activity due to a dosing at or above a minimum inhibitory concentration for antimicrobial activity. In some embodiments, the effective amount comprises an antimicrobial dose of the CMT derivative. The viral infection can be a coronavirus infection, such as but not limited to a COVID-19 infection.

The CMT derivative can be given at a dose of about 40 mg per day or less, optionally less than about 50 mg per day, less than about 60 mg per day, less than about 70 mg per day, less than about 80 mg per day, less than about 90 mg per day, less than about 100 mg per day, or less than about 200 mg per day. In some embodiments, an antimicrobial dose of the CMT derivative, such as doxycycline, is administered, such as 100 mg twice daily or daily. In some embodiments, the dosage is administered at 200 mg per day for one or more days, and then the dosage is reduced to 100 mg per day for one or more days. In some embodiments, the dose is administered over a period of days, such as ranging from about 5 to about 14 days. In some embodiments, the dose is administered for an extended period of time, such as for the duration of time in which a subject might be exposed to possible infection by a virus, such as a coronavirus.

The subject to be treated can be a human subject, such as but not limited to a human subject is suffering from a coronavirus infection, or at risk for exposure to a coronavirus infection. In some embodiments, the subject is at high risk for morbidity and mortality from SARS-CoV-2 infection. For example, in some embodiments, subjects are patients with pulmonary compromise, such as lung cancer, autoimmune disease, and/or pneumonia.

The CMT derivative can be configured to inhibit metalloproteinases (MMPs), optionally MMP-9, optionally any MMP implicated in respiratory distress syndrome (ARDS). The CMT derivative is configured to inhibit IL6 and other cytokine production. The CMT derivative is configured to inhibit Papain-like protease (PLpro), optionally wherein inhibition of PLpro prevents proteolytic cleavage of a replicase polyprotein needed for viral replication, optionally wherein the replicase polyprotein is non-structural proteins 1, 2 and/or 3 (Nsp1, Nsp2 and/or Nsp3). The CMT derivative can be configured to inhibit and/or bind 3C-like main protease (3CLpro) and/or Nsp5, optionally wherein inhibition and/or binding of 3CLpro and/or Nsp5 prevents viral replication. In some aspects, the CMT derivative can be configured to bind a divalent cation and transport the divalent cation intracellularly, optionally wherein transporting divalent cations intracellularly increases an intracellular concentration of the cation to thereby inhibit viral replication. Other features of the CMT derivative are described in the Examples.

Also provided herein are methods of treating a viral infection in a subject, including but not limited to, preventing, mitigating and/or prophylactically treating a viral infection in a subject, the method comprising administering to a subject in need an effective amount of a CMT derivative of the presently disclosed subject matter. In some embodiments, the effective amount comprises a dose of the CMT derivative sufficient to inhibit activation of gut microbiome inflammatory cells, wherein the dose of the CMT derivative is below a minimum inhibitory concentration for antimicrobial activity. In some embodiments, the CMT derivative can have antimicrobial activity due to a dosing at or above a minimum inhibitory concentration. In some embodiments, the effective amount comprises an antimicrobial dose of the CMT derivative. The viral infection can be a coronavirus infection, such as but not limited to a COVID-19 infection. In some embodiments, the CMT derivative is disposed in an effective amount in an excipient or a pharmaceutically acceptable carrier

In some embodiments, the CMT derivative can be minocycline, tigecycline, lymecycline, demeclocycline, oxytetracycline, rolitetracycline, or doxycycline. The CMT derivative can be given at a dose of about 40 mg per day or less, optionally less than about 50 mg per day, less than about 60 mg per day, less than about 70 mg per day, less than about 80 mg per day, less than about 90 mg per day, less than about 100 mg per day, or less than about 200 mg per day. In some embodiments, an antimicrobial dose of the CMT derivative, such as doxycycline, is administered, such as 100 mg twice daily or daily. In some embodiments, the dosage is administered at 200 mg per day for one or more days, and then the dosage is reduced to 100 mg per day for one or more days. In some embodiments, the dose is administered over a period of days, such as ranging from about 5 to about 14 days. In some embodiments, the dose is administered for an extended period of time, such as for the duration of time in which a subject might be exposed to possible infection by a virus, such as a coronavirus.

The subject can be a human subject, such as but not limited to a human subject is suffering from a coronavirus infection, or at risk for exposure to a coronavirus infection. In some embodiments, the subject is at high risk for morbidity and mortality from SARS-CoV-2 infection. For example, in some embodiments, subjects are patients with pulmonary compromise, such as lung cancer, autoimmune disease, and/or pneumonia.

Such methods can further comprise administering to the subject a dose of a divalent cation. In some embodiments, the divalent cation is Zn²⁺. In some embodiments, the divalent cation or Zn²⁺ is administered at a dosage of about 4 mg/day to about 50 mg/day, including at a dosage of about 4 mg/day to about 40 mg/day.

Provided herein are compositions for administration to a subject, the composition comprising a chemically modified tetracycline (CMT) derivative. In some embodiments, the CMT derivative lacks anti-microbial activity. In some embodiments, the CMT derivative can have antimicrobial activity due to a dosing at or above a minimum inhibitory concentration. In some embodiments the CMT derivative comprises a phenol ring, and/or comprises a chemical structure sufficient to chelate and/or bind a divalent cation, optionally Zn²⁺. In some embodiments, the CMT derivative is disposed in an effective amount in an excipient or a pharmaceutically acceptable carrier

Such compositions can further comprise a source of a divalent cation, optionally a source of Zn²⁺, optionally wherein the divalent cation or Zn²⁺ is included in the composition at a concentration sufficient to provide a dose of about 4 mg/day to about 50 mg/day when administered to a subject, including at a dosage of about 4 mg/day to about 40 mg/day.

The compositions can be configured to prevent, mitigate and/or prophylactically treat a viral infection, optionally a coronavirus infection, optionally a COVID-19 infection, when administered to a subject. The compositions can further comprise an excipient or a pharmaceutically acceptable carrier.

In some embodiments, the CMT derivative can be minocycline, tigecycline, lymecycline, demeclocycline, oxytetracycline, rolitetracycline, or doxycycline. The CMT derivative can be given at a dose of about 40 mg per day or less, optionally less than about 50 mg per day, less than about 60 mg per day, less than about 70 mg per day, less than about 80 mg per day, less than about 90 mg per day, less than about 100 mg per day, or less than about 200 mg per day. In some embodiments, the CMT derivative can have antimicrobial activity due to a dosing at or above a minimum inhibitory concentration. In some embodiments, an antimicrobial dose of the CMT derivative, such as doxycycline, is administered, such as 100 mg twice daily or daily. In some embodiments, the dosage is administered at 200 mg per day for one or more days, and then the dosage is reduced to 100 mg per day for one or more days. In some embodiments, the dose is administered over a period of days, such as ranging from about 5 to about 14 days. In some embodiments, the dose is administered for an extended period of time, such as for the duration of time in which a subject might be exposed to possible infection by a virus, such as a coronavirus.

In any of the above-discussed embodiments, the CMT derivative can comprise a compound having the following formula:

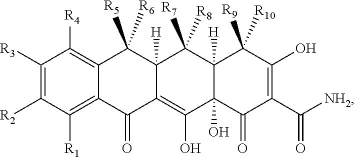

wherein,

each of R₁-R₄ are individually selected from the group consisting of H, hydroxy, halo, alkyl, alkoxy, amino, alkylamino, dialkylamino, and acylamino; and

each of R₅-R₁₀ is selected from H, hydroxy, halo, alkyl, alkoxy, amino, alkylamino, and dialkylamino; or

a pharmaceutically acceptable salt thereof.

Optionally, R₁ is H or hydroxy.

Optionally, R₂ is H or acylamino, further optionally wherein R₂ is NHC(═O)—R₁₁, wherein R₁₁ is alkyl or substituted alkyl, further optionally amino-substituted alkyl.

Optionally, R₄ is halo, further optionally chloro.

Optionally, R₁₀ is H or dialkylamino, further optionally dimethylamino.

Optionally, R₆ is H or alkyl, further optionally methyl.

Optionally, R₅ is H or hydroxy.

Optionally, R₈ is H or hydroxy.

A standard antimicrobial dose of doxycycline is typically 100 mg twice daily or daily. Doxycycline has a clear dosing split between its cellular cytotoxic/antimicrobial effects (e.g., >100 mg) and its anti-inflammatory/antiviral/pro-ionophoric effects (e.g., 40 mg). This is a consideration for low-dose doxycycline (20 mg twice daily) as a prophylactic treatment against COVID-19 infection. To elaborate, in some embodiments, the presently disclosed subject matter provides methods and compositions for chemoprophylaxis with doxycycline for prevention of SARS-CoV-2 infection and decrease in severity of disease of those subjects who become COVID-19 positive. In some embodiments, a subject is dosed at 20 mg BID doxycycline. In some embodiments, dosing is at 20 mg doxycycline in morning and evening (dosed approximately 12 hours apart). Low-dose, sub-microbial doxycycline can thus reduce the infection rate of COVID-19 in previously uninfected subjects and reduce the clinical severity of COVID-19 disease for those subjects that become infected.

Generic low-dose doxycycline (20 mg, twice a day) is a tetracycline derivative most frequently prescribed for treatment of inflammatory lesions of rosacea in adults. ORACEA® is a branded, sustained release form of low-dose doxycycline (30 mg immediate release, 10 mg delayed release) also approved for rosacea, but dosed once per day. Doxycycline hyclate tablets at 20 mg are commercially available under the registered trademark PERIOSTAT® and has structural formula:

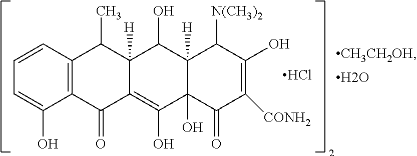

with an empirical formula of (C₂₂H₂₄N₂O₈.HCl).C₂H₆O.H₂O and a molecular weight of 1025.89. The chemical designation for doxycycline is 4-(dimethylamino)-1,4,4a,5,5a,6,11,12a-octahydro-3,5,10,12,12 apentahydroxy-6-methyl-1, 11-dioxo-2-naphthacenecarboxamide monohydrochloride, compound with ethyl alcohol (2:1), monohydrate.

Included within the scope of the presently disclosed subject matter are various individual anomers, diastereomers and enantiomers as well as mixtures thereof. In addition, the active compounds described herein also include any pharmaceutically acceptable salts, for example: alkali metal salts, such as sodium and potassium; ammonium salts; monoalkylammonium salts; dialkylammonium salts; trialkylammonium salts; tetraalkylammonium salts; and tromethamine salts. Hydrates, hyclates, and other solvates of the compounds are included within the scope of the presently disclosed subject matter.

In some embodiments, a composition of the presently disclosed subject matter can comprise one compound. In some embodiments, a composition of the presently disclosed subject matter can comprise more than one compound. In some embodiments, additional drugs or compounds useful for treating other disorders can be part of the composition. In some embodiments, a composition comprising only one compound can be administered at the same time as another composition comprising at least one other compound. In some embodiments, the different compositions can be administered at different times from one another. When a composition comprises only one compound, an additional composition comprising at least one additional compound can also be used.

The compositions of the presently disclosed subject matter can be administered by any route of administration reasonably expected to deliver the compositions to a desired target site in a subject. In some embodiments of the presently disclosed methods, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release.

The presently disclosed subject matter encompasses the preparation and use of pharmaceutical compositions comprising a compound useful for treatments as disclosed herein as an active ingredient. Such a pharmaceutical composition can consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a pharmaceutically acceptable ester or salt, such as in combination with a pharmaceutically acceptable cation or anion, as is well known in the art.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the presently disclosed subject matter is contemplated include, but are not limited to, humans and other primates, mammals including commercially relevant mammals or mammals kept as pets, such as cattle, pigs, horses, sheep, cats, and dogs, and birds including commercially relevant birds and birds kept as pets, such as chickens, ducks, geese, parrots, and turkeys.

Pharmaceutical compositions that are useful in the methods of the presently disclosed subject matter can be prepared, packaged, or sold in formulations suitable for oral, rectal, vaginal, parenteral, topical, pulmonary, intranasal, inhalation, buccal, ophthalmic, intrathecal or another route of administration.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject, or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, representative dose information is set forth herein above.

In addition to the active ingredient, a pharmaceutical composition of the presently disclosed subject matter can further comprise one or more additional pharmaceutically active agents. Particularly contemplated additional agents include anti-viral agents, anti-microbial agents, anti-emetics, scavengers (e.g., cyanide and cyanate scavengers), and pain relievers.

Controlled- or sustained-release formulations of a pharmaceutical composition of the presently disclosed subject matter can be made using conventional technology.

A formulation of a pharmaceutical composition of the presently disclosed subject matter suitable for oral administration can be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets can be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets can be non-coated or can be coated using known methods to achieve delayed disintegration in the gastrointestinal tract of a subject, thereby providing sustained release and absorption of the active ingredient. By way of example, a material such as glyceryl monostearate or glyceryl distearate can be used to coat tablets. Further by way of example, tablets can be coated using methods described in U.S. Pat. Nos. 4,256,108; 4,160,452; and U.S. Pat. No. 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Hard capsules comprising the active ingredient can be made using a physiologically degradable composition, such as gelatin. Such hard capsules comprise the active ingredient, and can further comprise additional ingredients including, for example, an inert solid diluent such as calcium carbonate, calcium phosphate, or kaolin.

Soft gelatin capsules comprising the active ingredient can be made using a physiologically degradable composition, such as gelatin. Such soft capsules comprise the active ingredient, which can be mixed with water or an oil medium such as peanut oil, liquid paraffin, or olive oil.

Lactulose can also be used as a freely erodible filler and is useful when the compounds of the presently disclosed subject matter are prepared in capsule form.

Liquid formulations of a pharmaceutical composition which are suitable for oral administration can be prepared, packaged, and sold either in liquid form or in the form of a dry product intended for reconstitution with water or another suitable vehicle prior to use.

Liquid suspensions can be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions can further comprise one or more additional ingredients including, but not limited to, suspending agents, dispersing or wetting agents, emulsifying agents, demulcents, preservatives, buffers, salts, flavorings, coloring agents, and sweetening agents. Oily suspensions can further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

In some embodiments, a preparation in the form of a syrup or elixir or for administration in the form of drops can comprise active ingredients together with a sweetener, which is preferably calorie-free, and which can further include methylparaben or propylparaben as antiseptics, a flavoring and a suitable color.

Liquid solutions of the active ingredient in aqueous or oily solvents can be prepared in substantially the same manner as liquid suspensions, the primary difference being that the active ingredient is dissolved, rather than suspended in the solvent. Liquid solutions of the pharmaceutical compositions can comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation can be prepared using known methods. Such formulations can be administered directly to a subject, used, for example, to form tablets, to fill capsules, or to prepare an aqueous or oily suspension or solution by addition of an aqueous or oily vehicle thereto. Each of these formulations can further comprise one or more of a dispersing or wetting agent, a suspending agent, and a preservative. Additional excipients, such as fillers and sweetening, flavoring, or coloring agents, can also be included in these formulations.

A pharmaceutical composition of the presently disclosed subject matter can also be prepared, packaged, or sold in the form of oil in water emulsion or a water-in-oil emulsion. The oily phase can be a vegetable oil such as olive or arachis oil, a mineral oil such as liquid paraffin, or a combination of these. Such compositions can further comprise one or more emulsifying agents including naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions can also contain additional ingredients including, for example, sweetening or flavoring agents.

A pharmaceutical composition of the presently disclosed subject matter can be prepared, packaged, or sold in a formulation suitable for rectal administration. Such a composition can be in the form of, for example, a suppository, a retention enema preparation, and a solution for rectal or colonic irrigation.

Suppository formulations can be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations can further comprise various additional ingredients including, but not limited to, antioxidants and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, and intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations can be prepared, packaged, or sold in a form suitable for bolus administration or for continuous administration. Injectable formulations can be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations can further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using a non-toxic parenterally acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil in water or water in oil emulsions such as creams, ointments or pastes, and solutions or suspensions. Formulations for topical administration can further comprise one or more of the additional ingredients described herein.

A pharmaceutical composition of the presently disclosed subject matter can be prepared, packaged, or sold in a formulation suitable for pulmonary administration via the buccal cavity. Such a formulation can comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant can be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant can constitute about 50% to about 99.9% (w/w) of the composition, and the active ingredient can constitute about 0.1% to about 20% (w/w) of the composition. The propellant can further comprise additional ingredients such as a liquid non-ionic or solid anionic surfactant or a solid diluent (preferably having a particle size of the same order as particles comprising the active ingredient).

Pharmaceutical compositions of the presently disclosed subject matter formulated for pulmonary delivery can also provide the active ingredient in the form of droplets of a solution or suspension. Such formulations can be prepared, packaged, or sold as aqueous or dilute alcoholic solutions or suspensions, optionally sterile, comprising the active ingredient, and can conveniently be administered using any nebulization or atomization device. Such formulations can further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the presently disclosed subject matter.

A pharmaceutical composition of the presently disclosed subject matter can be prepared, packaged, or sold in a formulation suitable for ophthalmic administration. Such drops can further comprise buffering agents, salts, or one or more other of the additional ingredients described herein. Other ophthalmically-administrable formulations which are useful include those which comprise the active ingredient in microcrystalline form or in a liposomal preparation.

A pharmaceutical composition of the presently disclosed subject matter can be prepared, packaged, or sold in a formulation suitable for intramucosal administration. The presently disclosed subject matter provides for intramucosal administration of compounds to allow passage or absorption of the compounds across mucosa. Such type of administration is useful for absorption orally (gingival, sublingual, buccal, etc.), rectally, vaginally, pulmonary, nasally, etc.

In some aspects, sublingual administration has an advantage for active ingredients which in some cases, when given orally, are subject to a substantial first pass metabolism and enzymatic degradation through the liver, resulting in rapid metabolization and a loss of therapeutic activity related to the activity of the liver enzymes that convert the molecule into inactive metabolites, or the activity of which is decreased because of this bioconversion.

In some cases, a sublingual route of administration is capable of producing a rapid onset of action due to the considerable permeability and vascularization of the buccal mucosa. Moreover, sublingual administration can also allow the administration of active ingredients which are not normally absorbed at the level of the stomach mucosa or digestive mucosa after oral administration, or alternatively which are partially or completely degraded in acidic medium after ingestion of, for example, a tablet.

Sublingual tablet preparation techniques known from the prior art are usually prepared by direct compression of a mixture of powders comprising the active ingredient and excipients for compression, such as diluents, binders, disintegrating agents and adjuvants. In an alternative method of preparation, the active ingredient and the compression excipients can be dry- or wet-granulated beforehand. In some embodiments, the active ingredient is distributed throughout the mass of the tablet. WO 00/16750 describes a tablet for sublingual use that disintegrates rapidly and comprises an ordered mixture in which the active ingredient is in the form of microparticles which adhere to the surface of water-soluble particles that are substantially greater in size, constituting a support for the active microparticles, the composition also comprising a mucoadhesive agent. WO 00/57858 describes a tablet for sublingual use, comprising an active ingredient combined with an effervescent system intended to promote absorption, and also a pH-modifier.

When a controlled-release pharmaceutical preparation of the presently disclosed subject matter further contains a hydrophilic base, many options are available for inclusion. Hydrophilic polymers such as a polyethylene glycol and polyvinyl pyrrolidone, sugar alcohols such as D-sorbitol and xylitol, saccharides such as sucrose, maltose, lactulose, D-fructose, dextran, and glucose, surfactants such as polyoxyethylene-hydrogenated castor oil, polyoxyethylene polyoxypropylene glycol, and polyoxyethylene sorbitan higher fatty acid esters, salts such as sodium chloride and magnesium chloride, organic acids such as citric acid and tartaric acid, amino acids such as glycine, beta-alanine, and lysine hydrochloride, and aminosaccharides such as meglumine are given as examples of the hydrophilic base. Polyethylene glycol, sucrose, and polyvinyl pyrrolidone are preferred and polyethylene glycol are further preferred. One or a combination of two or more hydrophilic bases can be used in the presently disclosed subject matter.

The presently disclosed subject matter contemplates pulmonary, nasal, or oral administration through an inhaler. In some embodiments, delivery from an inhaler can be a metered dose.

An inhaler is a device for patient self-administration of at least one active compound as described herein comprising a spray inhaler (e.g., a nasal, oral, or pulmonary spray inhaler) containing an aerosol spray formulation of at least one active compound and a pharmaceutically acceptable dispersant. In some embodiments, the device is metered to disperse an amount of the aerosol formulation by forming a spray that contains a dose of at least one active compound effective to treat a disease or disorder encompassed by the presently disclosed subject matter. The dispersant can be a surfactant, such as, but not limited to, polyoxyethylene fatty acid esters, polyoxyethylene fatty acid alcohols, and polyoxyethylene sorbitan fatty acid esters. Phospholipid-based surfactants also can be used.

In some embodiments, the aerosol formulation is provided as a dry powder aerosol formulation in which an active compound of the presently disclosed subject matter is present as a finely divided powder. The dry powder formulation can further comprise a bulking agent, such as, but not limited to, lactose, sorbitol, sucrose, and mannitol.

In some embodiments, the aerosol formulation is a liquid aerosol formulation further comprising a pharmaceutically acceptable diluent, such as, but not limited to, sterile water, saline, buffered saline and dextrose solution.

In some embodiments, the aerosol formulation further comprises at least one additional active compound as described herein in a concentration such that the metered amount of the aerosol formulation dispersed by the device contains a dose of the additional active compound in a metered amount that is effective to ameliorate the symptoms of disease or disorder disclosed herein when used in combination with at least a first or second active compound.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which can be included in the pharmaceutical compositions of the presently disclosed subject matter are known in the art and described, for example in Genaro, ed., 1985, Remington's Pharmaceutical Sciences, Mack Publishing Co., Easton, Pa., which is incorporated herein by reference.

The presently disclosed subject matter also includes a kit comprising the active ingredients(s) and an instructional material that describes administration of the ingredient(s). In some embodiments, this kit comprises a (preferably sterile) solvent suitable for dissolving or suspending the composition or compositions of the presently disclosed subject matter prior to administering the composition(s) to the mammal.

As used herein, an “instructional material” includes a publication, a recording, a diagram, or any other medium of expression that can be used to communicate the usefulness of the active compositions in the kit for effecting alleviation of the various diseases or disorders recited herein. Optionally, or alternately, the instructional material can describe one or more methods of alleviating the diseases or disorders. The instructional material of the kit of the presently disclosed subject matter can, for example, be affixed to a container that contains an active compound or compounds or be shipped together with a container that contains the compound or compounds. Alternatively, the instructional material can be shipped separately from the container with the intention that the instructional material and the active ingredient(s) be used cooperatively by the recipient.

When two or more active ingredients are to be administered, they can be administered in the same pharmaceutical composition or in separate pharmaceutical compositions. When administered in separate pharmaceutical compositions, they can be administered simultaneously, or one can be administered first. The amount of time between administration of the different active ingredients can vary and can be determined by one of ordinary skill in the art. For example, the two compounds could be administered up to 10 minutes apart, up to 30 minutes apart, up to 1 hour apart, etc. In some embodiments, one or more of the active ingredients can be administered more than once. In some embodiments, an active ingredient is administered at least twice. In some embodiments, the method is useful for low dose treatment. In some embodiments, the method is useful for short-term treatment.

### III. EXAMPLES

The presently disclosed subject matter will be now be described more fully hereinafter with reference to the accompanying Examples, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Infection with novel SARS-CoV-2 carries significant morbidity and mortality in patients with pulmonary compromise, such as lung cancer, autoimmune disease, and pneumonia. For early stages of mild to moderate disease, care is entirely supportive. Antiviral drugs such as remdesivir may be of some benefit but are reserved for severe cases given limited availability and potential toxicity.

Repurposing of safer, established medications that have antiviral activity is an approach for treatment of earlier-stage disease. Tetracycline and its derivatives (e.g. doxycycline and minocycline) are nontraditional antibiotics with a well-established safety profile, potential efficacy against viral pathogens such as dengue fever and chikungunya, and regulate pathways important in initial infection, replication, and systemic response to SARS-CoV-2. The following Examples present a series of four high-risk, symptomatic, COVID-19+ patients, with known pulmonary disease, treated with doxycycline with subsequent rapid clinical improvement. No safety issues were noted with use of doxycycline.

Doxycycline is an attractive candidate as a repurposed drug in the treatment of COVID-19 infection, with an established safety profile, strong preclinical rationale, and compelling initial clinical experience described here.

In view of the above considerations, the following Examples report a series of four patients with COVID-19 infection and known high-risk pulmonary disease who were placed on standard doses of doxycycline for a course between 5 and 14 days. Patients were initiated on therapy with doxycycline after discussion of potential risks and benefits and after informed consent was obtained. There was no concomitant use of any other antibiotics, antiviral agents, antimalarial drugs, zinc, or any supplements postulated to be beneficial in therapy for SARS-CoV-2 in these four patients.

### Example 1

A 48-year-old black woman, never smoker, with stage IV epidermal growth factor receptor (EGFR) mutated adenocarcinoma of lung, diagnosed in August 2018, was treated with osimertinib since November 2018 with response in all sites of disease. Other medications included loperamide and doxycycline, as needed for EGFR inhibitor-related diarrhea and rash, respectively. She worked as a nursing assistant. On 6 Apr. 2020 she reported fever of 101° F., headache, anorexia and anosmia. On examination: temperature 101° F., pulse 102, blood pressure 115/71, O2 sat 98% on room air. She was noted to have mild cough with normal respiratory effort. Laboratory tests revealed white blood cell count of 2.7, with lymphopenia, similar to prior recent determinations; C-reactive protein (CRP) 10.6 (0-5 mg/l), creatine kinase (CK) 2996 (46-179 U/l), troponin <0.006 (negative), aspartate aminotransferase 105 (13-39 U/l), creatinine 1.3 (05-1.1) mg/dl. Chest X-ray showed chronic stable right retrocardiac midlung opacities with no new focal consolidation or effusion. The following day her swab returned positive for SARS-CoV-2. As she had declined hospitalization, she was instructed to discontinue osimertinib but to start doxycycline initially 200 mg followed by 100 mg daily for 5 days. She noted improvement in all symptoms within 5 days.

### Example 2

A 71-year-old white male physician with a 9-year history of pulmonary sarcoidosis, treated with intermittent steroids, noted new intermittent cough and one episode of diarrhea in early April 2020. Nasopharyngeal swab was positive for SARS-CoV-2. No additional evaluation was performed. The patient was started on doxycycline 100 mg orally twice a day and self-monitored pulse oximetry at home. Revisit to the emergency department was prompted by transient drop in oxygen saturation to 88% on day six. His temperature was 100.4° F., chest X-ray showed evidence of his chronic lower lung changes attributed to sarcoidosis, 02 sat was 96%. The patient returned home without supplemental oxygen and remained on doxycycline for 10 days. Fever resolved and he remained asymptomatic. After 3 weeks retesting was negative for SARS-CoV-2 and he returned to work.

### Example 3

A 40-year-old Asian woman, never smoker, with stage IV EGFR mutated lung adenocarcinoma, on osimertinib since 13 Jun. 2018, also treated with stereotactic radiation to right lower lobe primary lesion in July 2019, presented to an urgent care clinic with 4 days of anosmia, mild cough, and mild dyspnea. She worked as a personal care assistant for elderly patients at home. Nasopharyngeal swab testing was positive for SARS-CoV-2 RNA. The patient continued to take osimertinib and doxycycline 100 mg twice daily (previously prescribed for EGFR-I related rash). Symptoms improved within 1 week. Repeat nasopharyngeal swab testing on 9 May 2020 was negative for SARS-CoV-2 RNA.

### Example 4

An 88-year-old white man, with several comorbidities including sick sinus syndrome, cardiovascular disease, lacunar strokes, mild obstructive lung disease, and chronic indwelling bladder catheter, was admitted from a Veterans Nursing Home with fever to 102° F., severe dry cough, weakness, and dyspnea. Chest X-ray showed bilateral diffuse infiltrates. Laboratory tests were notable for nasopharyngeal swab positive for SARS-CoV-2 RNA, CRP 11, mild hypoxia with O2 sat 89-92%. Ceftriaxone, azithromycin and 2 liters of nasal prong O2 were begun. His initial hospital course showed progression of his pulmonary symptomatology with decreasing O2 saturation requiring 4 liters of nasal prong O2 by day 3, with CRP increase to 13. On day 4 ceftriaxone was discontinued and azithromycin was replaced with doxycycline 100 mg twice daily. His pulmonary course quickly improved alongside decreasing CRP. By day 8, he no longer required oxygen and his CRP was 1.0. He never received any antiviral, IL-6 antibody, nor convalescent plasma. He was discharged on day 28 after his lung infiltrates resolved on chest X-ray after 14 days of doxycycline therapy.

### Discussion of Examples

These reported cases are the first to provide a correlative relationship between doxycycline and potential reduction of severity of symptoms in COVID-19+ patients. The patients reported here had a number of high-risk features, predictive of severe disease and increased risk of mortality. Patients one and four had advanced lung cancer on treatment, a group for whom mortality rates with COVID-19 infection is in excess of 33%.² Elevated inflammatory markers, including CRP, transaminases, and CK (as in cases 1 and 4) are also associated with worse outcomes (Chen et al., Ann Clin Microbiol Antimicrob 2020; 19: 18). Advanced age and diffuse bilateral infiltrates (case 4), are also predictors of high morbidity and mortality (Du et al., Eur Respir J 2020; 55: 2000524). Case 4 demonstrated a temporal relationship between administration of doxycycline and clinical improvement, after several days of worsening on azithromycin. Patients with active pulmonary sarcoidosis, as in case 2, are also at greater risk of complications from COVID-19 due to immunologic dysfunction and dysregulation (Sweiss, et al., Chest. Epub ahead of print 29 Apr. 2020). The described patients were at a significant increased risk from COVID-19 morbidity and mortality. All improved, with one showing resolution of bilateral pulmonary infiltrates. None progressed to severe disease. A standard antimicrobial dose of doxycycline (100 mg twice daily or daily) was administered to the four patients in this case series. Doxycycline has a clear dosing split between its cellular cytotoxic/antimicrobial effects (>100 mg) and its potential anti-inflammatory/antiviral/pro-ionophoric effects (40 mg) (Griffin et al., Am J Physiol Cell Physiol 2010; 299:C539-C548; Fredeking et al., Recent Pat Antiinfect Drug Discov 2015; 10: 51-58; Kumar et al., J Virol 2008; 82: 9880-9889; Sargiacomo C, Sotgia F and Lisanti M P. Aging (Albany N.Y.) 2020; 12: 6511-6517; Conforti et al., Dermatol Ther 2020: e13437). This is a consideration for low-dose doxycycline (20 mg twice daily) as a prophylactic treatment against COVID-19 infection.

While it is not desired to be bound by any particular theory of operation, potential efficacy of doxycycline against COVID-19 may be due to pleiotropic effects against the general pathways involved in viral infection, replication, and associated over-exuberant inflammatory response, with associated angiogenic effects (Griffin, et al., Am J Physiol Cell Physiol 2010; 299:C539-C548). Doxycycline has no known direct specificity for inhibition of SARS-CoV-2. Doxycycline likely has activity against known coreceptors DPP4/CD26, through demonstrated inhibition of NF-κB,14,15 and coreceptor CD147/EMMPRIN, necessary for entry of SARS-CoV into T lymphocytes, even at submicrobial doses (Emingil, et al., J Periodontol 2008; 79:469-476; Wang et al., Cell Mol Immunol. Epub ahead of print 7 Apr. 2020. DOI: 10.1038/s41423-020-0424-9DPP4 appears to have a binding site for NF-κB and inhibition of NF-κB decreases DPP4 expression (Choi et al., Circulation 2017; 135: 1935-1950). DPP4 demonstrates increased expression in older patients and those with diabetes or pulmonary disease, and may account in part for increased morbidity and mortality to COVID-19 (Sargiacomo C, Sotgia F and Lisanti M P. Aging (Albany N.Y.) 2020; 12: 6511-6517). Finally, MMP-9 also appears to be required for the virus to traverse the cell wall for infection (Kong et al. Viruses 2015; 7: 4230-4253; Phillips et al., J Virol 2017; 91: e01564-16.). Once COVID-19 has infected the cell, structural analysis indicates doxycycline may be able to bind to PLpro, which is responsible for proteolytic cleavage of the replicase polyprotein that releases nonstructural proteins 1, 2 and 3 (Nsp1, Nsp2 and Nsp3), essential for viral replication (Wu et al., Acta Pharm Sin B 2020; 10: 766-788). It is similarly predicted to bind to 3CLpro or Nsp5, which is cleaved from the polyproteins, causing further cleavage of Nsp4-16, mediating maturation of Nsps essential in the virus lifecycle (Wu et al., Acta Pharm Sin B 2020; 10: 766-788).

Doxycycline is an ionophore that binds divalent cations (including zinc) to facilitate cell membrane transport, and thereby increase zinc concentration in the cell (Griffin et al., Am J Physiol Cell Physiol 2010; 299:C539-C548; te Velthuis et al., PLoS Pathog 2010; 6: e1001176)) Elevated zinc has an inhibitory effect on the replication of SARSCoV-2 (te Velthuis et al., PLoS Pathog 2010; 6: e1001176). The presumed mechanisms are through inhibition of proper processing of replicase proteins and RNA-dependent RNA polymerase (RdRp) activity (te Velthuis et al., PLoS Pathog 2010; 6: e1001176).

Lastly, doxycycline may help prevent the “cytokine storm” following COVID-19 infection presumed to be due to hyperactivity of the immune response to SARS-CoV-2. A primary driver of the anti-inflammatory activity of doxycycline is secondary to its direct inhibition of NF-κB (Santa-Cecilia et al., Neurotox Res 2016; 29: 447-459). NF-κB directly regulates IL-6 expression, which is a key driver of the cytokine storm (Brasier, Cardiovasc Res 2010; 86:211-218). Doxycycline may also precipitate apoptosis of senescent epithelial cells (Sargiacomo et al., Aging (Albany N.Y.) 2020; 12: 6511-6517), which have known increased expression of DPP4/CD26, likely exhibiting increased viral replication in these cells which leads to induction of the cytokine storm.

In summary, these Examples present reports of four patients at high risk for morbidity and mortality from SARS-CoV-2 infection who experienced improvement and/or mild clinical course in association with doxycycline. Despite the safety of doxycycline, this medication should be used for treatment of COVID-19+ patients, under direct physician guidance and monitoring.

### REFERENCES

All references listed below, as well as all references cited in the instant disclosure (including the Examples), including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
1. Centers for Disease Control and Prevention. Coronavirus disease 2019 (COVID-19) treatment guidelines, www.cdc.gov/coronavirus/2019-ncov/need-extra-precautions/people-at-higher-risk. html (Accessed 21 May 2020).
2. Garassino M. TERAVOLT (Thoracic cancERs international coVid 19 cOLlaboraTion): first results of a global collaboration to address the impact of COVID-19 in patients with thoracic malignancies. Presented at the American Association for Cancer Research Annual Virtual Meeting, 20-22 Jul. 2020, Philadelphia, Pa.
3. Griffin M O, Fricovsky E, Ceballos G, et al. Tetracyclines: a pleiotropic family of compounds with promising therapeutic properties. Review of the literature. Am J Physiol Cell Physiol 2010; 299:C539-C548.
4. Fredeking T M, Zavala-Castro J E, González-Martinez P, et al. Dengue patients treated with doxycycline showed lower mortality associated to a reduction in IL-6 and TNF levels. Recent Pat Antiinfect Drug Discov 2015; 10: 51-58.
5. Rothan H A, Bahrani H, Mohamed Z, et al. A combination of doxycycline and ribavirin alleviated chikungunya infection. PLoS One 2015; 10: e0126360.
6. Malek A E, Granwehr B P and Kontoyiannis D P. Doxycycline as a potential partner of COVID-19 therapies. IDCases 2020; 21: e00864.
7. Kumar N, Xin Z-T, Liang Y, et al. NF-kappaB signaling differentially regulates influenza virus RNA synthesis. J Virol 2008; 82: 9880-9889.
8. Sargiacomo C, Sotgia F and Lisanti M P. COVID-19 and chronological aging: senolytics and other anti-aging drugs for the treatment or prevention of corona virus infection? Aging (Albany N.Y.) 2020; 12: 6511-6517.
9. Wu C, Liu Y, Yang Y, et al. Analysis of therapeutic targets for SARS-CoV-2 and discovery of potential drugs by computational methods. Acta Pharm Sin B 2020; 10: 766-788.
10. Kong M Y F, Whitley R J, Peng N, et al. Matrix metalloproteinase-9 mediates RSV infection in vitro and in vivo. Viruses 2015; 7: 4230-4253.
11. Phillips J M, Gallagher T and Weiss S R. Neurovirulent murine coronavirus JHM.SD uses cellular zinc metalloproteases for virus entry and cell-cell fusion. J Virol 2017; 91: e01564-16.
12. Conforti C, Giuffrida R, Zalaudek I, et al. Doxycycline, a widely used antibiotic in dermatology with a possible anti-inflammatory action against IL-6 in COVID-19 outbreak. Dermatol Ther 2020: e13437.
13. te Velthuis A J W, van den Worm S H E, Sims A C, et al. Zn2+ inhibits coronavirus and arterivirus RNA polymerase activity in vitro and zinc ionophores block the replication of these viruses in cell culture. PLoS Pathog 2010; 6: e1001176.
14. Santa-Cecilia F V, Socias B, Ouidja M O, et al. Doxycycline suppresses microglial activation by inhibiting the p38 MAPK and NF-kB signaling pathways. Neurotox Res 2016; 29: 447-459.
15. Choi B, Lee S, Kim S-M, et al. Dipeptidyl peptidase-4 induces aortic valve calcification by inhibiting insulin-like growth factor-1 signaling in valvular interstitial cells. Circulation 2017; 135: 1935-1950.
16. Emingil G, Atilla G, Sorsa T, et al. The effect of adjunctive subantimicrobial dose doxycycline therapy on GCF EMMPRIN levels in chronic periodontitis. J Periodontol 2008; 79:469-476.
17. Wang X, Xu W, Hu G, et al. SARS-CoV-2 infects T lymphocytes through its spike protein mediated membrane fusion. Cell Mol Immunol. Epub ahead of print 7 Apr. 2020. DOI: 10.1038/s41423-020-0424-9.
18. Chen W, Zheng K I, Liu S, et al. Plasma CRP level is positively associated with the severity of COVID-19. Ann Clin Microbiol Antimicrob 2020; 19: 18.
19. Du R-H, Liang L-R, Yang C-Q, et al. Predictors of mortality for patients with COVID-19 pneumonia caused by SARS-CoV-2: a prospective cohort study. Eur Respir J 2020; 55: 2000524.
20. Sweiss N J, Korsten P, Syed H J, et al. When the game changes: guidance to adjust sarcoidosis management during the COVID-19 pandemic. Chest. Epub ahead of print 29 Apr. 2020. DOI: 10.1016/j.chest.2020.04.033.
21. Brasier A R. The nuclear factor-kappaBinterleukin-6 signalling pathway mediating vascular inflammation. Cardiovasc Res 2010; 86:211-218.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of preventing, mitigating and/or prophylactically treating a viral infection in a subject, the method comprising administering to a subject in need thereof a dose of a chemically modified tetracycline (CMT) derivative, wherein the CMT derivative comprises a phenol ring, and comprises a chemical structure sufficient to chelate and/or bind a divalent cation, wherein the dose of the CMT derivative is below a minimum inhibitory concentration for antimicrobial activity.

2. The method of claim 1, wherein the viral infection is a coronavirus infection, optionally a COVID-19 infection.

3. The method of claim 1 or claim 2, wherein the CMT derivative lacks antimicrobial activity, optionally wherein the CMT derivative lacks antimicrobial activity due to deletion of a C4 dimethylamino.

4. The method of claim 1 or claim 2, wherein the phenol ring comprises a diethylamino group to enhance scavenging of reactive oxygen species.

5. The method of claim 1 or claim 2, wherein the chemical structure comprises the following structure:

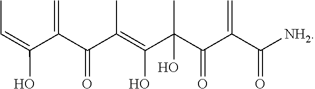 [SMILES: C=C(C(N)=O)C(=O)C(C)(O)/C(O)=C(\C)C(=O)C(=C)/C(O)=C\C]

6. The method of claim 1 or claim 2, wherein the CMT derivative is doxycycline.

7. The method of any of the above claims, wherein the CMT derivative is given at a dose of about 40 mg per day or less, optionally less than about 50 mg per 30 day, less than about 60 mg per day, less than about 70 mg per day, less than about 80 mg per day, less than about 90 mg per day, or less than about 100 mg per day.

8. The method of any of the above claims, wherein the CMT derivative is configured to inhibit metalloproteinases (MMPs), optionally MMP-9, optionally any MMP implicated in respiratory distress syndrome (ARDS).

9. The method of any of the above claims, wherein the CMT derivative is configured to inhibit IL6 and other cytokine production.

10. The method of any of the above claims, wherein the CMT derivative is configured to inhibit Papain-like protease (PLpro), optionally wherein inhibition of PLpro prevents proteolytic cleavage of a replicase polyprotein needed for viral replication, optionally wherein the replicase polyprotein is non-structural proteins 1, 2 and/or 3 (Nsp1, Nsp2 and/or Nsp3).

11. The method of any of the above claims, wherein the CMT derivative is configured to inhibit and/or bind 3C-like main protease (3CLpro) and/or Nsp5, optionally wherein inhibition and/or binding of 3CLpro and/or Nsp5 prevents viral replication.

12. The method of any of the above claims, wherein the CMT derivative is configured to bind a divalent cation and transport the divalent cation intracellularly, optionally wherein transporting divalent cations intracellularly increases an intracellular concentration of the cation to thereby inhibit viral replication.

13. The method of any of the above claims, wherein the subject is a human subject, optionally wherein the human subject is suffering from a coronavirus infection.

14. The method of any of the above claims, further comprising administering to the subject a dose of a divalent cation, optionally Zn²⁺, optionally wherein the divalent cation or Zn²⁺ is administered at a dosage of about 4 mg/day to about 50 mg/day.

15. A composition for administration to a subject, the composition comprising a chemically modified tetracycline (CMT) derivative, wherein the CMT derivative optionally lacks anti-microbial activity, comprises a phenol ring, and comprises a chemical structure sufficient to chelate and/or bind a divalent cation, optionally Zn²⁺, and wherein the CMT derivative is included in the composition at a concentration sufficient to provide a dose of about 40 mg per day or less, optionally less than about 50 mg per day, less than about 60 mg per day, less than about 70 mg per day, less than about 80 mg per day, less than about 90 mg per day, or less than about 100 mg per day when administered to a subject.

16. The composition of claim 15, further comprising a source of a divalent cation, optionally a source of Zn²⁺, optionally wherein the divalent cation or Zn²⁺ is included in the composition at a concentration sufficient to provide a dose of about 4 mg/day to about 40 mg/day when administered to a subject.

17. The composition of claim 15 or claim 16, wherein the composition is configured to prevent, mitigate and/or prophylactically treat a viral infection, optionally a coronavirus infection, optionally a COVID-19 infection, when administered to a subject.

18. The composition of any of claims 15-17, wherein the CMT derivative lacks antimicrobial activity due to deletion of a C4 dimethylamino.

19. The composition of any of claims 15-18, wherein the CMT derivative lacks antimicrobial activity due to dosing below a minimum inhibitory concentration.

20. The composition of any of claims 15-17 and 19, wherein the CMT derivative is doxycycline.

21. The composition of any of claims 15-20, further comprising an excipient or a pharmaceutically acceptable carrier.

22. The composition of any of claims 15-21, wherein the CMT derivative is configured to inhibit metalloproteinases (MMPs), optionally MMP-9, optionally any MMP implicated in respiratory distress syndrome (ARDS).

23. The composition of any of claims 15-22, wherein the CMT derivative is configured to inhibit IL6 and other cytokine production.

24. The composition of any of claims 15-23, wherein the CMT derivative is configured to inhibit Papain-like protease (PLpro), optionally wherein inhibition of PLpro prevents proteolytic cleavage of a replicase polyprotein needed for viral replication, optionally wherein the replicase polyprotein is non-structural proteins 1, 2 and/or 3 (Nsp1, Nsp2 and/or Nsp3).

25. The composition of any of claims 15-24, wherein the CMT derivative is configured to inhibit and/or bind 3C-like main protease (3CLpro) and/or Nsp5, optionally wherein inhibition and/or binding of 3CLpro and/or Nsp5 prevents viral replication.

26. The composition of any of claims 15-25, wherein the CMT derivative is configured to bind a divalent cation and transport the divalent cation intracellularly, optionally wherein transporting divalent cations intracellularly increases an intracellular concentration of the cation to thereby inhibit viral replication.

27. A chemically modified tetracycline (CMT) derivative for the prevention, mitigation and/or prophylactic treatment of a viral infection, wherein the CMT derivative comprises a phenol ring; and comprises a chemical structure sufficient to chelate and/or bind a divalent cation.

28. The CMT derivative of claim 27, wherein the CMT derivative can have antimicrobial activity due to a dosing at or above a minimum inhibitory concentration.

29. The CMT derivative of claim 27 or claim 28, wherein the viral infection is a coronavirus infection, optionally a COVID-19 infection.

30. The CMT derivative of claim 27 or claim 29, wherein the CMT derivative lacks anti-microbial activity.

31. The CMT derivative of claim 30, wherein the CMT derivative lacks antimicrobial activity due to deletion of a C4 dimethylamino or wherein the CMT derivative lacks antimicrobial activity due to dosing below a minimum inhibitory concentration.

32. The CMT derivative of any of claims 27-31, wherein the phenol ring comprises a diethylamino group to enhance scavenging of reactive oxygen species.

33. The CMT derivative of any of claims 27-32, wherein the chemical structure comprises the following structure:

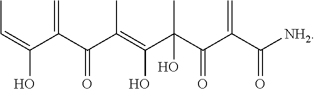 [SMILES: C=C(C(N)=O)C(=O)C(C)(O)/C(O)=C(\C)C(=O)C(=C)/C(O)=C\C]

34. The CMT derivative of any of claims 27-33, wherein the CMT derivative is doxycycline.

35. The CMT derivative of any of claims 27-34, wherein the CMT is disposed in an effective amount in an excipient or a pharmaceutically acceptable carrier.

36. The CMT derivative of any of claims 27-35, wherein the CMT derivative is configured to inhibit metalloproteinases (MMPs), optionally MMP-9, optionally any MMP implicated in respiratory distress syndrome (ARDS).

37. The CMT derivative of any of claims 27-36, wherein the CMT derivative is configured to inhibit IL6 and other cytokine production.

38. The CMT derivative of any of claims 27-37, wherein the CMT derivative is configured to inhibit Papain-like protease (PLpro), optionally wherein inhibition of PLpro prevents proteolytic cleavage of a replicase polyprotein needed for viral replication, optionally wherein the replicase polyprotein is non-structural proteins 1, 2 and/or 3 (Nsp1, Nsp2 and/or Nsp3).

39. The CMT derivative of any of claims 27-38, wherein the CMT derivative is configured to inhibit and/or bind 3C-like main protease (3CLpro) and/or Nsp5, optionally wherein inhibition and/or binding of 3CLpro and/or Nsp5 prevents viral replication.

40. The CMT derivative of any of claims 27-39, wherein the divalent cation is Zn²⁺.

41. The CMT derivative of any of claims 27-40, wherein the CMT derivative is configured to bind a divalent cation and transport the divalent cation intracellularly, optionally wherein transporting divalent cations intracellularly increases an intracellular concentration of the cation to thereby inhibit viral replication.

42. A method for the prevention, mitigation and/or prophylactic treatment of a viral infection in a subject, the method comprising administering to a subject in need thereof an effective amount of a chemically modified tetracycline (CMT) derivative, wherein the CMT derivative comprises a phenol ring, and comprises a chemical structure sufficient to chelate and/or bind a divalent cation.

43. The method of claim 42, wherein the CMT derivative can have antimicrobial activity due to a dosing at or above a minimum inhibitory concentration.

44. The method of claim 42 or claim 43, wherein the viral infection is a coronavirus infection, optionally a COVID-19 infection.

45. The method of any of claims 42-44, wherein the phenol ring comprises a diethylamino group to enhance scavenging of reactive oxygen species.

46. The method of any of claims 42-45, wherein the CMT comprises the following structure:

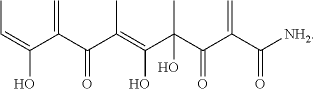 [SMILES: C=C(C(N)=O)C(=O)C(C)(O)/C(O)=C(\C)C(=O)C(=C)/C(O)=C\C]

47. The method of any of claims 42-46, wherein the CMT derivative is doxycycline.

48. The method of any of claims 42-47, wherein the CMT derivative is configured to inhibit metalloproteinases (MMPs), optionally MMP-9, optionally any MMP implicated in respiratory distress syndrome (ARDS).

49. The method of any of claims 42-48, wherein the CMT derivative is configured to inhibit IL6 and other cytokine production.

50. The method of any of claims 42-49, wherein the CMT derivative is configured to inhibit Papain-like protease (PLpro), optionally wherein inhibition of PLpro prevents proteolytic cleavage of a replicase polyprotein needed for viral replication, optionally wherein the replicase polyprotein is non-structural proteins 1, 2 and/or 3 (Nsp1, Nsp2 and/or Nsp3).

51. The method of any of claims 42-50, wherein the CMT derivative is configured to inhibit and/or bind 3C-like main protease (3CLpro) and/or Nsp5, optionally wherein inhibition and/or binding of 3CLpro and/or Nsp5 prevents viral replication.

52. The method of any of claims 42-51, wherein the CMT derivative is configured to bind a divalent cation and transport the divalent cation intracellularly, optionally wherein transporting divalent cations intracellularly increases an intracellular concentration of the cation to thereby inhibit viral replication.

53. The method of any of claims 42-52, wherein the subject is a human subject, optionally wherein the human subject is suffering from a coronavirus infection.

54. The method of any of claims 42-53, further comprising administering to the subject a dose of a divalent cation, optionally Zn²⁺, optionally wherein the divalent cation or Zn²⁺ is administered at a dosage of about 4 mg/day to about 40 mg/day.

55. A chemically modified tetracycline (CMT) derivative for the prevention, mitigation and/or prophylactic treatment of a viral infection, wherein the CMT derivative:
comprises a phenol ring; and
comprises a chemical structure sufficient to chelate and/or bind a divalent cation, optionally Zn²⁺.
